(19) United States
(12) Patent Application Publication
(10) Pub. No.: US 2017/0265749 A1
(43) Pub. Date: Sep. 21, 2017

(54) PROCESSING APPARATUS AND PROCESSING METHOD

(71) Applicant: CANON KABUSHIKI KAISHA, Tokyo (JP)

(72) Inventor: Nobuhito Suehira, Tokyo (JP)

(21) Appl. No.: 15/446,196

(22) Filed: Mar. 1, 2017

(30) Foreign Application Priority Data
Mar. 15, 2016 (JP) 2016-051615

(51) Int. Cl. A61B 5/00; G01J 3/02; G01J 3/44; A61B 5/02; G01N 29/24; G01N 29/46; G01N 29/06; G01J 3/28; G01J 3/42

(52) U.S. Cl. CPC A61B 5/0095; G01J 3/2889; G01J 3/0289; G01J 3/4412; G01J 3/42; G01N 29/2418; G01N 29/46; G01N 29/0654; A61B 5/02007; G01J 2003/283

(57) ABSTRACT

A processing apparatus, comprises: a first acquirer configured to acquire a first specific information distribution of an object based on acoustic waves propagating from the object onto which light is irradiated; a second acquirer configured to acquire a characteristic value of the first specific information distribution of the object; a third acquirer configured to acquire information indicating a correspondence between an optical coefficient and the characteristic value of the first specific information distribution; and a fourth acquirer configured to acquire the optical coefficient of the object using the characteristic value of the first specific information distribution of the object and the information indicating the correspondence.

### BACKGROUND OF THE INVENTION

Field of the Invention

The present invention relates to a processing apparatus and a processing method.

Description of the Related Art

Clinical application of apparatuses that estimate optical coefficient information (such as optical absorption coefficients, effective scattering coefficients, and effective attenuation coefficients) on objects such as living bodies has been proposed. In addition, as a method for measuring optical coefficient information on objects, time-resolved spectroscopy (TRS) described in QUANTITATIVE MEASUREMENT OF OPTICAL PARAMETERS IN NORMAL BREASTS USING TIME-RESOLVED SPECTROSCOPY: IN-VIVO RESULTS OF 30 JAPANESE WOMEN, Kazunori Suzuki, Journal of Biomedical optics 1 (3), 330-334 (July 1996) or the like has been proposed.

### SUMMARY OF THE INVENTION

A method described in QUANTITATIVE MEASUREMENT OF OPTICAL PARAMETERS IN NORMAL BREASTS USING TIME-RESOLVED SPECTROSCOPY: IN-VIVO RESULTS OF 30 JAPANESE WOMEN, Kazunori Suzuki, Journal of Biomedical optics 1 (3), 330-334 (July 1996) requires a photodetector to acquire optical coefficient information on objects. However, it may be desirable to acquire optical coefficient information on objects without a photodetector.

The present invention has been made in view of the above problem and has an object of providing a method of acquiring optical coefficient information on objects in place of the method of using a photodetector.

An embodiment of the present invention provides a processing apparatus including: a first acquirer configured to acquire a first specific information distribution of an object based on an electrical signal acquired by receiving acoustic waves propagating from the object onto which light is irradiated; a second acquirer configured to acquire a characteristic value of the first specific information distribution of the object; a third acquirer configured to acquire information indicating a correspondence between an optical coefficient and the characteristic value of the first specific information distribution; and a fourth acquirer configured to acquire the optical coefficient of the object using the characteristic value of the first specific information distribution of the object and the information indicating the correspondence.

An embodiment of the present invention provides a processing method comprising: a first acquisition step of acquiring a first specific information distribution of an object based on an electrical signal acquired by receiving acoustic waves propagating from the object onto which light is irradiated; a second acquisition step of acquiring a characteristic value of the first specific information distribution of the object; a third acquisition step of acquiring information indicating a correspondence between an optical coefficient and the characteristic value of the first specific information distribution; and a fourth acquisition step of acquiring the optical coefficient of the object using the characteristic value of the first specific information distribution of the object and the information indicating the correspondence.

According to an embodiment of the present invention, it is possible to simply acquire background optical coefficients of objects.

Further features of the present invention will become apparent from the following description of exemplary embodiments with reference to the attached drawings.

### BRIEF DESCRIPTION OF THE DRAWINGS

FIGS. 1A to 1C are photoacoustic images of phantoms having different optical coefficients;

FIGS. 2A and 2B show an example of the configuration of a processing apparatus in a first embodiment;

FIG. 3 is a flowchart showing an example of the operation of the processing apparatus in the first embodiment;

FIGS. 4A and 4B show a projection image in a second embodiment;

FIG. 5 is a flowchart showing an example of the operation of the processing apparatus in the second embodiment;

FIGS. 6A and 6B show an example of the configuration of the processing apparatus in the third embodiment; and

FIG. 7 is a view showing a state in which a light irradiation position is scanned in the third embodiment.

### DESCRIPTION OF THE EMBODIMENTS

Hereinafter, a description will be given of the preferred embodiments of the present invention with reference to the drawings. However, the dimensions, materials, shapes, their relative arrangements, or the like of constituents that will be described below may be appropriately changed depending on the configurations or various conditions of apparatuses to which the present invention is applied. Accordingly, the dimensions, materials, shapes, their relative arrangements, or the like of the constituents do not intend to limit the scope of the invention to the following descriptions.

The present invention relates to a technology for acquiring optical coefficient information on an object based on acoustic waves propagating from the object. The optical coefficient information on the object includes a representative value of the optical coefficients of the object and distribution information indicating optical coefficients at a plurality of positions inside the object. As the representative value, an average value, a central value, or the like of the optical coefficients inside the object may be employed. In the specification, a representative value of the optical coefficients of the object will be called a background optical coefficient of the object. The optical coefficients include at least one of a light absorption coefficient, a light scattering coefficient, and a light attenuation coefficient. The present invention is grasped as a processing apparatus, a control method for the processing apparatus, a processing method, an object information acquiring method, or a signal processing method. In addition, the present invention is also grasped as a program that causes an information processing apparatus having hardware resources such as a CPU and a memory to perform these methods, or grasped as a storage medium storing the program.

The processing apparatus of the present invention includes a photoacoustic apparatus using a photoacoustic effect in which acoustic waves generated inside an object by irradiating light (electromagnetic waves) onto the object are received to acquire specific information on the object as image data. In this case, the specific information is information on characteristic values corresponding to a plurality of positions inside the object, the information being generated using a reception signal obtained by receiving photoacoustic waves.

Specific information acquired by photoacoustic measurement is a value reflecting an absorption ratio of optical energy. For example, the specific information includes the generation source of acoustic waves generated by light irradiation, initial sound pressure inside an object, optical energy absorption density or an absorption coefficient derived from initial sound pressure, and substance concentration constituting tissues. It is possible to calculate an oxygen saturation distribution by the calculation of oxyhemoglobin concentration and deoxyhemoglobin concentration as the substance concentration. In addition, it is also possible to calculate glucose concentration, collagen concentration, melanin concentration, a volume fraction of fat or water, or the like.

Based on specific information on respective positions inside an object, a two-dimensional or three-dimensional specific information distribution is acquired. Distribution data may be generated as image data. The specific information may be calculated as distribution information on respective positions inside the object rather than being calculated as numerical data. That is, the specific information is distribution information such as an initial sound pressure distribution, an energy absorption density distribution, an absorption coefficient distribution, and an oxygen saturation distribution. Three-dimensional (or two-dimensional) image data indicates a reconstruction-unit specific information distribution arranged in three-dimensional (or two-dimensional) space. The reconstruction unit corresponds to a voxel in the case of a three dimension and corresponds to a pixel in the case of a two dimension.

In the present invention, acoustic waves are typically ultrasound waves and include elastic waves called sound waves or acoustic waves. An electrical signal converted from acoustic waves by a probe or the like is called an acoustic signal. However, in the specification, the ultrasound waves or acoustic waves do not intend to limit wavelengths of such elastic waves. The acoustic waves generated by the photoacoustic effect are called photoacoustic waves. An electrical signal derived from the photoacoustic waves is also called a photoacoustic signal. In addition, in the specification, a technology for imaging the specific information based on the photoacoustic measurement will be called photoacoustic tomography.

### First Embodiment

(Principle) The principle of the present invention will be described. First, sound pressure (P) generated when light is irradiated onto an absorber is expressed by formula (1).

[Math. 1]

P=Γ·μₐ₀·Φ (1)

Γ is a Gruneisen coefficient indicating an elasticity specific value and obtained by dividing a volume expansion coefficient (β) and the square of the speed of sound (c) by specific heat (Cp). μₐ₀ is an absorption coefficient of the absorber and is not a background optical coefficient. Φ is an light intensity (intensity of the light irradiated onto the absorber) in a local region. Φ is also called light fluence.

The light intensity Φ is expressed by, for example, formula (2) using a depth function z.

[Math. 2]

Φ=Φ₀EXP(−μ_{eff}·z) (2)

Φ₀ is incident light on the surface of an object. Accordingly, formula (2) indicates that the light exponentially attenuates as it travels in a depth direction. Note that μ_{eff} is an average effective attenuation coefficient inside a medium, reflects a scattering coefficient and an absorption coefficient, and is included in a background optical coefficient.

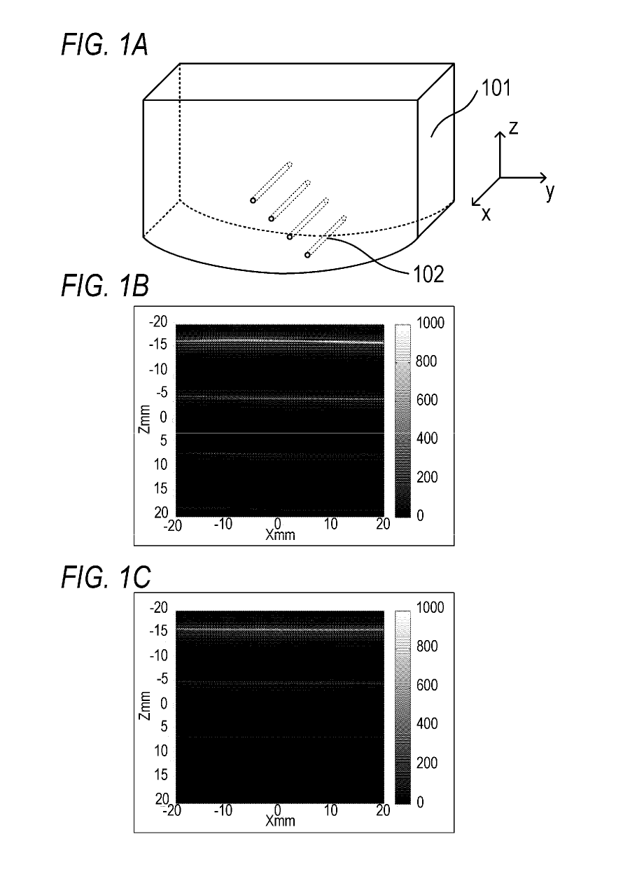

Next, FIGS. 1B and 1C show images of a phantom 101 taken by a processing apparatus. The processing apparatus has probes arranged on a hemispherical face as will be described in a third embodiment, and irradiates light while scanning with its XY plane. The light is irradiated onto the phantom 101 from a negative Z direction. Note that since a reconstruction image is obtained by adding up data in a certain region calculated for each scanning position, it may be said that the substantially-parallel light is irradiated in a direction substantially parallel to the Z-axis direction of the phantom 101. That is, the light reflecting an effective attenuation coefficient attenuates only in the depth direction. Note that a distribution is also generated in XY directions in the case of a point light source. That is, a light distribution reflecting a scattering coefficient and an absorption coefficient is generated.

FIG. 1A shows the structure of the phantom 101. The base material of the phantom 101 is a urethane resin, and absorbers and scatterers for adjusting a background optical coefficient are distributed in the base material. In order to conduct an experiment, two types of urethane resins were used. A first phantom has a background absorption coefficient of 0.002/mm and a scattering coefficient of 0.4/mm. A second phantom has a background absorption coefficient of 0.004/mm and a scattering coefficient of 0.8/mm. These values are ranges assuming human skins. Each of the phantoms includes nylon wires having an absorption coefficient of 0.1/mm and a thickness of 1.0 mm as targets. The optical coefficient is a value close to that of human vessels. In addition, the targets are arranged by four in total at positions away from each other by 10 mm in a Y direction and a Z direction.

FIG. 1B shows a photoacoustic image of the first phantom in which a maximum value is projected in the Y direction and the first to third targets from the surface are visually recognizable. FIG. 1C is a photoacoustic image of the second phantom in which a maximum value is projected in the Y direction. In the image, the third target from the surface is hardly recognizable. The first phantom has a smaller background optical coefficient than that of the second phantom, whereby the targets at deeper positions are made visually recognizable.

As described above, signal ranges reflect the background optical coefficients. Note here that the signal ranges indicate ranges in which the targets are visually recognizable in the photoacoustic images in which the maximum values are projected. On the other hand, since the above theoretical formulae (1) and (2) are based on some hypotheses, experiments and results may be different from each other. Therefore, in case that a database in which experimental values and background optical coefficients are associated with each other is created, it is possible to obtain more accurate background optical coefficients.

(Apparatus Configuration)

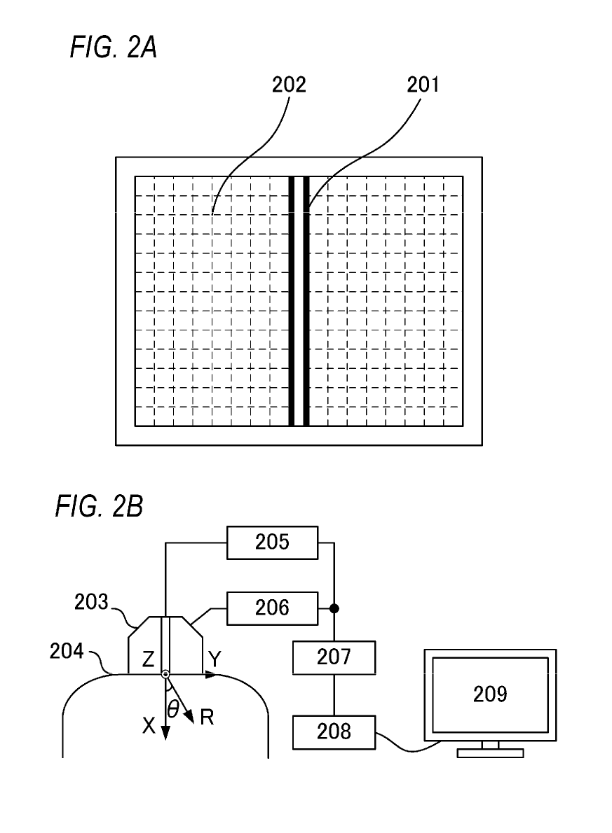

As an example of the processing apparatus of the present invention, a description will be given of an apparatus using a hand-held photoacoustic probe. FIG. 2A shows the arrangements of a probe and a light irradiation unit in the hand-held photoacoustic probe. The line-shaped light irradiation unit 201 is arranged at the center, and a two-dimensional probe 202 is arranged on both sides of the line-shaped light irradiation unit 201.

FIG. 2B shows the configuration of the processing apparatus. The apparatus is constituted by the hand-held photoacoustic probe 203 described above, a light control unit 205, a signal processing unit 206, an apparatus control unit 207, an information processing unit 208, and a display unit 209. The photoacoustic probe 203 is arranged so as to make its probe surface contact an object 204. In the processing apparatus, photoacoustic measurement is made possible by synchronizing light irradiated from the light irradiation unit 201 with the reception timing of the probe 202.

The apparatus control unit 207 gives instructions to perform control on the entire apparatus such as the control of a light source and the reception control of the probe. In addition, the apparatus control unit 207 is provided with a user interface (UI) and allowed to perform a change in measurement parameters, start and end of measurement, selection of an image processing method, storage of patients' information and images, analysis of data, or the like, based on instructions from an operator. The information processing unit 208 performs information processing such as image reconstruction. Further, obtained images are displayed on the display unit 209.

The light irradiation unit 201 is a line-shaped part that irradiates pulsed light onto the object 204. The pulsed light is transmitted from the light source to the light irradiation unit 201 via an optical system (not shown) The optical system includes, for example, optical devices such as a lens, a mirror, a prism, an optical fiber, and a diffusion plate. In addition, in guiding the light, a shape and density of the light may be changed using such optical devices to obtain a desired light distribution. Note that the intensity (maximum allowable exposure amount) of the light allowed to be irradiated onto a unit area is fixed as a standard on the irradiation of the laser light or the like onto living body tissues. In order to satisfy the standard, it is only necessary to expand the light by a certain degree. In the embodiment, the pulsed light is introduced from the light source to the light irradiation unit 201 by a bundle fiber. That is, a plurality of point light sources is arranged in a line to form the line-shaped light source. Note that the structure of the irradiation unit is not limited to this. The light may be expanded by a lens or the like to form the line-shaped light source through a slit. In addition, the light is irradiated in a line shape in order to generate a two-dimensional tomogram here but may be configured to be irradiated onto a wide region of the object.

As the light source, it is preferable to use a laser light source to obtain a large output. However, a light-emission diode, a flash lamp, or the like may be used. In the case of using a laser, various types such as a solid-state laser, a gas laser, a dye laser, and a semiconductor laser may be used. The irradiation timing, waveform, intensity, or the like of the light is controlled by the light control unit 205.

In addition, in order to effectively generate photoacoustic waves, it is necessary to irradiate the light in a substantially short period of time according to the heat characteristics of the object. When the object is a living body, the pulsed light generated from the light source preferably has a pulse width of about 10 to 50 nanoseconds. In addition, the pulsed light preferably has a wavelength at which the light propagates through the inside of the object. Specifically, for a living body, the pulsed light has a wavelength of 700 nm or more and 1100 nm or less. Since the light in this region reaches a relatively deep part of a living body, it is possible to acquire information on the deep part of the living body. When only the surface part of a living body is measured, visible light having a wavelength of about 500 to 700 nm to a near-infrared region may be used. Moreover, a wavelength of the pulsed light preferably has a high absorption coefficient depending on an observation target. Here, a titanium sapphire laser serving as a solid-state laser is used as the light source and has a wavelength of 760 nm and 800 nm. When the irradiation of light having a plurality of wavelengths is configured to be allowed, it is possible to calculate substance density using a difference in the degree of absorption for each of the wavelengths.

The probe 202 receives acoustic waves propagating from the object onto which the light has been irradiated and outputs an electrical signal. The probe 202 preferably has high reception sensitivity for photoacoustic waves generated by the object and has a wide frequency band. The two-dimensional probe 202 is a device that performs the reception of photoacoustic waves and the transmission/reception of ultrasound waves, and is also called a transducer. Examples of such a device include a PZT (Piezoelectric Ceramic) and a CMUT (Capacitive Micro Machine Probe). One side of the hand-held probe 202 of the embodiment is constituted by, for example, 64×10 devices. The devices receive acoustic waves and output an electrical signal. The electrical signal converted by the probe 202 is transmitted to the signal processing unit 206. Note that the reception timing of the acoustic waves is controlled by the apparatus control unit 207 so as to be synchronized with the irradiation of the light. The probe 202 has a band of 2 to 5 MHz. When an ultrasound wave transceiver that will be described later is used, it may also serve as the probe 202. Alternatively, the ultrasound wave transceiver may be separately used for each of light acoustic measurement and ultrasound wave measurement depending on a central frequency.

The signal processing unit 206 performs the signal processing of the electrical signal received from the probe 202. The signal processing unit 206 performs the filtering of the electrical signal described above, amplification, and the generation of a digital signal through A/D conversion, and transmits the generated digital signal to the apparatus control unit 207. In addition, 2048 sampling is performed at a sampling frequency of 40 MHz. Data is 12-bit data with a code. The signal processing unit 206 is typically constituted by an OP amplifier, an A/D converter, a FPGA, an ASIC, or the like.

The information processing unit 208 generates the distribution of specific information at respective positions inside the object using the electrical signal received from the signal processing unit 206. More specifically, the information processing unit 208 generates a photoacoustic image inside the object by image reconstruction using a photoacoustic signal derived from photoacoustic waves. Besides, the information processing unit 208 has information processing performance necessary for light intensity calculation and background optical coefficient acquisition. In addition, when acquiring ultrasound wave attenuation characteristics by ultrasound wave measurement, the information processing unit 208 processes an ultrasound wave signal derived from an ultrasound wave echo. The information processing unit 208 further performs desired processing such as signal correction. The information processing unit 208 may be constituted by an information processing apparatus including a processor, a memory, or the like. The respective functions of the information processing unit 208 are implemented when the processor runs a program stored in the memory. However, some or all of the functions of the information processing unit 208 may be implemented by a circuit such as an ASIC and a FPGA. In addition, the information processing unit 208 may be constituted by an information processing apparatus common to the light control unit 205 and the apparatus control unit 207. Note that the signal processing unit 206 and the information processing unit 208 may be constituted by a plurality of devices or circuits. The information processing unit 208 is preferably a computer, a workstation, or the like. Note that in the specification, the respective functions of the information processing unit 208 will also be described as acquirers.

(Image Reconstruction)

Image reconstruction is performed by the information processing unit 208. The image reconstruction is, for example, three-dimensional image reconstruction in which the information processing unit 208 reconstructs a three-dimensional image from an electrical signal output from a probe and then acquires a desired specific information distribution from the three-dimensional image. The image reconstruction uses a known reconstruction method such as universal back-projection and phasing addition. Here, a method using the universal back-projection will be described. An initial sound pressure distribution p(r) is expressed by formula (3).

$$\begin{matrix}[ Math . 3 ] & \\ P ( r ) = {\int }_{{Ω}_{0}} b ( {r}_{0} , t = \vert r - {r}_{0} \vert ) \frac{d {Ω}_{0}}{{Ω}_{0}} & ( 3 )\end{matrix}$$

At this time, a term b(r₀,t) corresponding to projection data is expressed by formula (4).

$$\begin{matrix}[ Math . 4 ] & \\ b ( {r}_{0} , t ) = 2 {p}_{d} ( {r}_{0} , t ) - 2 t \frac{\partial {p}_{d} ( {r}_{0} , t )}{\partial t} & ( 4 )\end{matrix}$$

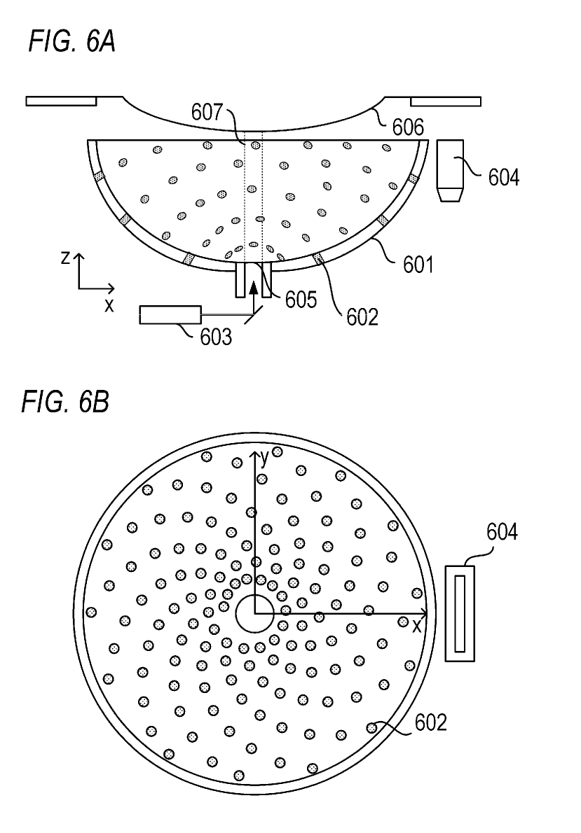

Here, p_{d}(r₀,t) is a photoacoustic signal detected by a detection device, r₀ is the position of each detection device, t is a time, and Ω₀ is a solid angle of the probe. As a photoacoustic image, an initial sound pressure distribution as described above or an energy absorption density distribution stipulated by an initial sound pressure and an absorption coefficient may be used. Note that image precision is preferably corrected since it changes depending on distributed places or directions (angles) of vessels inside an object. For example, when a probe is arranged in a hemispherical container, resolution in performing imaging is higher near the center of a hemisphere but lower toward the periphery of the hemisphere. Therefore, in forming a photoacoustic image, it is only necessary to correct a peripheral region with measurement data at a plurality of places or the like. In addition, in the case of a hemispherical container as shown in FIG. 6A, the signal becomes weaker as an angle formed with a Z axis becomes smaller. Therefore, it may also be possible to perform processing to enhance the intensity of the signal of absorbers according to an angle with the Z axis.

(Database)

Information (hereinafter called a “correspondence”) in which a photoacoustic image and a background optical coefficient are associated with each other is stored as a part of a database in a storage unit not shown of the processing apparatus. The database may be created by collecting, for example, data on clinical studies or actual clinical fields. Since the same segments have, of course, the same structures as in vessels, it is preferable to create the database for each of the segments. Note that it may also be possible to construct the database in a storage unit separately from the processing apparatus and cause the processing apparatus to access the database where necessary.

(Processing Flow)

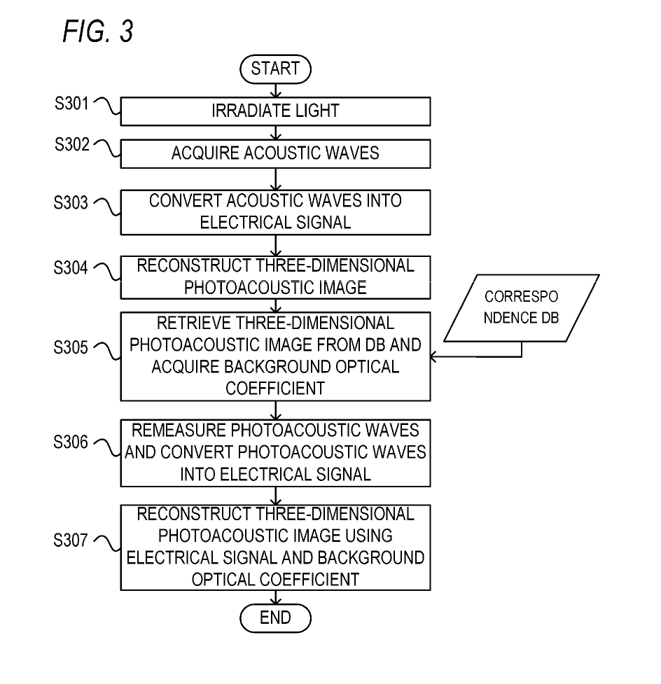

A description will be given, with reference to a flowchart shown in FIG. 3, of the flow of the operation of the processing apparatus in the embodiment.

In step S301, an object is irradiated with pulsed light emitted from a light source. The pulsed light incident on the inside of the object is absorbed by specific absorbers corresponding to a wavelength of the pulsed light. The absorbers having absorbed the light expand and contract to generate acoustic waves over their surrounding areas.

In step S302, the probe 202 receives the acoustic waves generated by the absorbers inside the object. In step S303, the probe 202 converts the acquired acoustic waves into an electrical signal and outputs the electrical signal to the signal processing unit 206. In the signal processing unit 206, the input analog electrical signal is amplified, and acquired at a prescribed sampling frequency and converted into a digital signal by an A/D converter. After that, the signal processing unit 206 outputs the digital signal to the information processing unit 208.

In step S304, the information processing unit 208 generates a photoacoustic image inside the object based on a photoacoustic signal derived from the photoacoustic waves acquired by the probe 202. Note that the photoacoustic image here is generated using an initial sound pressure distribution, an absorption coefficient distribution, or an optical energy absorption density distribution (first specific information distribution). At this time, the information processing unit 208 operates as a first acquirer of the present invention. When the absorption coefficient distribution is used at this point, a light intensity distribution is calculated by a temporary background optical coefficient such as a general statistical value. Here, the light intensity distribution is associated with a position and intensity of the irradiated light, the surface shape of the object, and an optical coefficient distribution inside the object. Therefore, the processing apparatus is preferably configured to include a camera to take an image of the surface shape of the object, or the probe is preferably configured to acquire the surface shape based on an ultrasound wave echo. In addition, as will be described later, a holding member that holds the object to stipulate the surface shape is preferably provided.

In step S305, the information processing unit 208 acquires a background optical coefficient based on the correspondence between the background optical coefficient and a characteristic value of the first specific information distribution (for example, an optical energy absorption density distribution). Here, the information processing unit 208 acquires the background optical coefficient inside the object based on the image reconstructed in step S304 and the correspondence stored in the storage unit as a database. More specifically, the information processing unit 208 first acquires information indicating the correspondence between an optical coefficient and a specific information distribution from, for example, the database. At this time, the information processing unit 208 operates as a third acquirer of the present invention. Subsequently, the information processing unit 208 retrieves the image reconstructed in step S304 from the database using, for example, a pattern recognition technology to acquire the background optical coefficient of the object. At this time, the information processing unit 208 operates as a fourth acquirer of the present invention. In this case, a pattern appearing in the reconstructed image is also recognized as a characteristic value of first specific information. In addition, like the “signal range” of the phantom described above, the target visually-recognizable range of a photoacoustic image where a maximum value is projected is also recognized as the characteristic value of the first specific information. As described above, when the characteristic value of the first specific information is acquired, the information processing unit 208 operates as the third acquirer of the present invention. The correspondence between the reconstructed image and the background optical coefficient may be the database of image data and background optical coefficients acquired from a multiplicity of objects at, for example, clinical fields or the like or may be a relational expression or the like of parameters and background optical coefficients extracted from the reconstructed image. Note that a scattering coefficient greatly contributes to the spread of the photoacoustic signal in a direction (substantially orthogonal direction, a typically perpendicular direction that will be called a “perpendicular direction”) different from a direction (hereinafter called an “irradiation direction”) in which the light is incident on the object from a light source. In addition, an absorption coefficient and a scattering coefficient contribute to the spread of the photoacoustic signal in the irradiation direction.

In step S306, the processing apparatus performs the remeasurement of photoacoustic waves to acquire a photoacoustic signal again. Since a digital signal is acquired from the photoacoustic waves in the same procedure as steps S301 to S303, its description will be omitted. Alternatively, instead of the remeasurement, it may also be possible to store in advance the results measured in steps S301 to S303 in the storage unit of the processing apparatus and read the data in step S306. In this case, since it is only necessary to perform the photoacoustic measurement in the processing of the flowchart shown in FIG. 3 once, the processing becomes simpler as a whole. In addition, it may also be possible to perform correction calculation using an accurately-obtained background optical coefficient.

In step S307, the information processing unit 208 acquires the light intensity distribution of the light inside the object using a background optical coefficient distribution, and acquires an absorption coefficient distribution (second specific information distribution) inside the object using the light intensity distribution and a photoacoustic measurement result acquired in step S306. At this time, the information processing unit 208 operates as a fifth acquirer of the present invention.

According to the processing of the embodiment, it is possible to easily acquire a background optical coefficient from image data on a photoacoustic image without using a measurement device such as a spectroscope. In addition, an absorption coefficient distribution (second specific information distribution) based on a corrected light intensity distribution is acquired using the acquired background optical coefficient. In addition, accuracy in a background optical coefficient is further improved with an increase in the number of the correspondences of a database.

### Second Embodiment

(Method for Creating Database)

Hereinafter, a description will be given of a method for creating a database in a second embodiment. Note that since the apparatus configuration of the second embodiment is the same as that of the first embodiment, its description will be omitted. In addition, the following description will be given with an assumption that a photoacoustic image has been obtained in the same procedure as steps S301 to S304 of the first embodiment.

The second embodiment is characterized in that a characteristic value extracted from the photoacoustic image is used to determine a background optical coefficient. In the second embodiment, a database storing the correspondence between a characteristic value and a background optical coefficient is used. Prior to the targeted photoacoustic measurement of an object, it is necessary to prepare for the database described above. The following procedure aims to create the database and targets at a multiplicity of objects. Alternatively, it may also be possible to create the database based on measurement data acquired from an object himself/herself who is a target to be finally measured.

As an example of the measurement, it is assumed that the light irradiation unit 201 has a line-shaped irradiation region as shown in FIG. 2A. Using the light irradiation unit 201 described above, the processing apparatus of the second embodiment creates cross-sectional images perpendicular to the line at prescribed intervals.

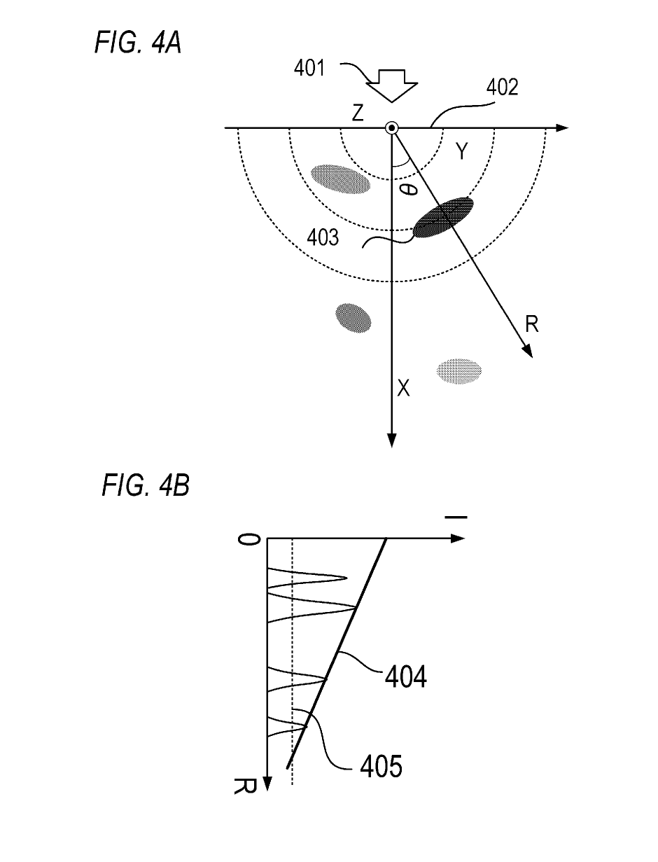

First, the information processing unit 208 generates an image in which only vessels having a desired thickness are extracted from a photoacoustic image. In addition, as shown in FIG. 4A, a cylindrical coordinate system is employed in which the line-shaped light irradiation unit 401 is defined as a Z axis and a distance from the light source is defined as R. Further, a projection image (Maximum Intensity Projection image) is created in which the maximum signal intensity of the photoacoustic image is projected in a Z direction. Processing for creating the MIP image may be implemented in such a manner that a maximum value is extracted from the corresponding positions of the plurality of cross-sectional images perpendicular to the line. Thus, an absorber 403 is projected, whereby it is possible to acquire a two-dimensional map.

Moreover, as shown in FIG. 4B, the maximum signal intensity may be projected with respect to an R axis. Thus, a maximum signal at an equal distance from the light irradiation unit 401 as indicated by dotted lines in FIG. 4A is acquired. However, a value of an angle θ formed with an X axis may be restricted to restrict a range of a projected signal. This is because an error may increase since living body tissues could have anisotropy. In addition, a range of the R axis of a signal having a threshold 405 or more is calculated as data. For example, an envelope line 404 is calculated from a maximum signal intensity projection image shown in FIG. 4B, and the distance between the intersection between the envelope line 404 and the threshold 405 and an origin is set as the range. The distance will be called a “specific information distance.” Thus, the specific information distance is extracted as a characteristic value from each object. Note that the envelope line 404 may be consequently one calculated from a signal from the same type of an absorber in formula (1). This is because a wavelength at which a strong signal is generated is different depending on an absorber and only an absorber that generates a strong signal is selected. Of course, it may also be possible to select a wavelength at which the signal of the same intensity is output from a different absorber. For example, in the case of an artery and a vein, signal intensity becomes the same when light near 800 nm is irradiated.

Note that a method for creating a photoacoustic image is not limited to the above one. For example, a photoacoustic image may be created by two-dimensional image data or three-dimensional volume data. In addition, the shape of the light irradiation unit 201 is not limited to a line shape. That is, point irradiation or surface irradiation may be performed.

Next, a description will be given of a method for acquiring a background optical coefficient. An optical coefficient such as an absorption coefficient and a scattering coefficient corresponding to an object may be measured by, for example, a spectroscopy system (NIRS) using near-infrared light. According to measurement based on the spectroscopy system, two fibers are, for example, used. First, pulsed light is irradiated onto a living body from one fiber, and then the light propagating through the living body is received by the other fiber. Further, the time response and frequency response of the received light are analyzed to calculate an absorption coefficient and a scattering coefficient. The measurement is preferably performed before or after photoacoustic measurement. When the light attenuates only in the Z direction as shown in FIGS. 1B and 1C or when the light attenuates only in the R direction as shown in FIGS. 4A and 4B, it may also be possible to use a conversion formula to further calculate an effective attenuation coefficient appearing in formula (1) from an absorption coefficient μₐ and a scattering coefficient μₛ measured by the spectroscopy system. Although the conversion formula for an effective attenuation coefficient μ_{eff} is different depending on a model but may be expressed by formula (5) using, for example, an anisotropic scattering parameter g.

[Math. 5]

μ_{eff}=\sqrt{3μₐ(1−g)μₛ} (5)

Note that the method for acquiring a background optical coefficient is not limited to the above one. Any appropriate method may be used according to a type of a light source or the like.

Further, a background optical coefficient of the same object and a specific information distance calculated from a photoacoustic image are associated with each other and stored in a database. In the manner described above, the database of a specific information distance and an optical coefficient may be created. Specifically, for example, the storage unit (not shown) of the processing apparatus stores the correspondence between a specific information distance and a background optical coefficient as a table or a mathematical formula. However, since it is not possible to collect all data, insufficient data may be interpolated by a phantom or simulation. In addition, the database may be constituted by data obtained by acquiring correspondences from a multiplicity of objects in advance and applying statistical processing to the acquired correspondences. Thus, the reliability of the data is improved, and mathematical processing is made possible. Thus, a background optical coefficient and a specific information distance indicating a characteristic value extracted from a photoacoustic image are associated with each other as a correspondence to prepare for a database in advance.

Note that in the embodiment, a specific information distance calculated from a projection image in which maximum signal intensity is projected is used as information associated with a background optical coefficient. However, other methods may be used. For example, a two-dimensional or three-dimensional image calculated from initial sound pressure or the like may be used. In this case, it is possible to verify such an image against a multiplicity of images stored in the storage unit by a similarity determination, pattern recognition, or the like. In addition, it may also be possible to digitize an index from the image, calculate in advance a formula in which the numerical value and a background optical coefficient are associated with each other, and calculate a background optical coefficient using the formula.

(Processing Flow)

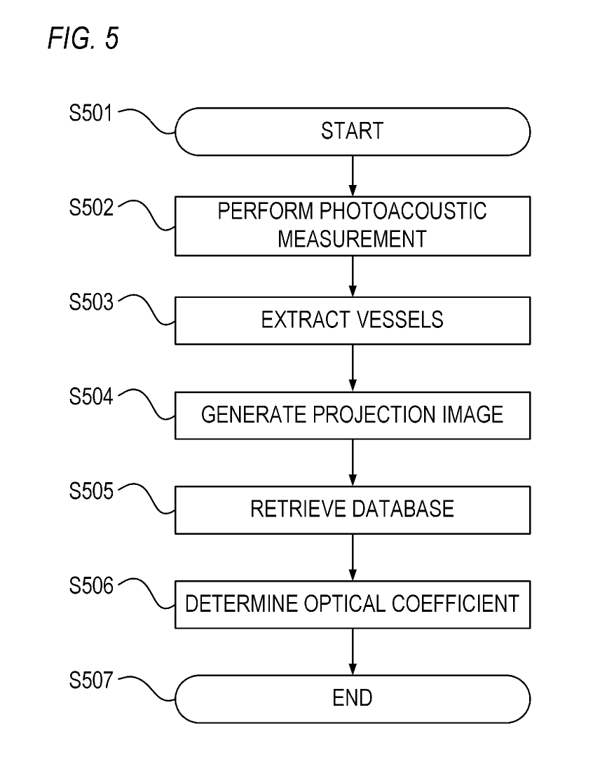

FIG. 5 is a flowchart showing a procedure for determining a background optical coefficient according to the second embodiment. The following measurement is performed on a new object different from an object involved in creating a database.

In step S501, measurement is started. In this state, an operator holds the photoacoustic probe 203 and brings the probe 202 into contact with the object via acoustic matching gel.

In step S502, photoacoustic measurement is performed. In synchronization with the irradiation of pulsed light from the light irradiation unit 201, the probe 202 receives photoacoustic waves. By performing the photoacoustic measurement while changing a wavelength of the pulsed light, it is possible to selectively form an image of arteries or veins.

In step S503, vessels are extracted from a photoacoustic image. In this step, absorbers having desired shapes may be extracted even if there are absorbers having different shapes such as vessels and tumors. Here, vessels having a thickness in a constant range (0.5 mm to 1.0 mm) are extracted as desired absorbers. The extraction of the desired absorbers aims to reduce the influence of a difference in frequency band contained in photoacoustic waves depending on the shapes of absorbers and the frequency characteristics of the sensitivity of the probe 202. For the extraction of vessels, a general method may be used. For example, a threshold for pixel values is determined for binarization, and regions in which a signal exists are recognized as vessels. In addition, an image filter such as a band pass filter may be used to obtain vessels having the same thickness.

In step S504, a specific information distance is calculated from a vessel image. The specific information distance is a distance from the origin to the intersection between the envelope line 404 and the threshold 405 in the one-dimensional projection image described above in FIG. 4B. Note that a value at the origin of the envelope line 404 is likely to be different depending on the color of a skin. In this case, the value may be corrected based on intensity near the origin located at the surface of the skin.

In step S505, a background optical coefficient corresponding to the specific information distance calculated in step S504 is retrieved from the database. In this case, the background optical coefficient is an effective attenuation coefficient, and a value closest to the specific information distance and a value second closest to the specific information distance are calculated.

When a specific information distance corresponding to the range of an error is found in step S506, an effective attenuation coefficient corresponding to the value is set as the background optical coefficient of the second embodiment. Alternatively, by interpolating the values between effective attenuation coefficients corresponding to respective specific information distances, it is possible to calculate a desired effective attenuation coefficient at the specific information distance acquired in step S504. The interpolation may be performed based on, for example, linear interpolation or polynomial interpolation.

In step S507, the measurement is finished.

The background optical coefficient of the object thus acquired may be used to calculate an light intensity distribution. Further, the light intensity distribution may be used to calculate an absorption coefficient distribution from an initial sound pressure distribution. The initial sound pressure distribution used at this time may be one acquired in step S502, or may be acquired by newly performing photoacoustic measurement. Alternatively, the acquired optical coefficient may be used to correct a photoacoustic image that has been generated. The second embodiment is advantageous in that the number of measurement times in the flowchart of FIG. 5 is only once.

As described above, according to the present invention, it is possible to simply calculate a background optical coefficient indicating the scattering and absorption of light inside an object by performing calculation using a photoacoustic image. In addition, it is possible to expect the calculation of a background optical coefficient having high reproducibility by calculating a specific information distance from vessels having a constant thickness. As a result, accuracy in reconstructing a photoacoustic image is also improved.

### Third Embodiment

(Apparatus Configuration)

FIGS. 6A and 6B show the probe portion of a processing apparatus that measures a breast in a third embodiment. FIG. 6A is a cross-sectional view of the probe portion of the processing apparatus. FIG. 6B is a plan view when probes are seen from their top surfaces.

First, a description will be given of the probe portion of the processing apparatus. Along the inner surface of a hemispherical container 601, the probes 602 are spirally arranged by 512. In addition, the hemispherical container 601 has, at its bottom part, space 605 where measurement light from a light irradiation unit 603 passes through. Further, the measurement light is irradiated onto an object from the negative direction of a Z axis. The object is placed on a holding member 606. The holding member 606 preferably uses a material that has intensity enough to support the object like polyethylene terephthalate and allows light and acoustic waves to pass through. Inside the hemispherical container 601 and the holding member 606, an acoustic matching material is filled where necessary. The acoustic matching material fills up the space between the object and the holding member 606 and the space between the holding member 606 and the probes 602 to acoustically connect the object and the probes 602 to each other. The acoustic matching material in each of the space may be different. The acoustic matching material preferably uses a material that has acoustic impedance close to those of the object and the probes 602 and in which the attenuation of acoustic waves is small. In addition, the acoustic matching material preferably causes pulsed light to pass through. For example, water, ricinus, gel, or the like may be used.

The relative positional relationship between the hemispherical container 601 and the object is changed by a scanning stage (not shown). The scanning stage changes the relative position of the hemispherical container 601 with respect to the object in X, Y, and Z directions. The scanning stage includes a guiding mechanism in the X, Y, and Z directions, a driving mechanism in the X, Y, and Z directions, and a position sensor that measures positions of the hemispherical container 601 in the X, Y, and Z directions. Typically, the hemispherical container 601 is mounted on the scanning stage. Therefore, it is preferable to use a linear guide or the like capable of withstanding a heavy load for the guiding mechanism. In addition, the driving mechanism is allowed to use a lead screw mechanism, a link mechanism, a gear mechanism, a hydraulic mechanism, or the like. As a driving force, a motor or the like may be used. In addition, as the position sensor, an optical or magnetic encoder or the like may be used.

Further, at respective positions at which the hemispherical container 601 is scanned by the scanning stage, substantially parallel pulsed light 607 is irradiated. The probes 602 are devices that detect photoacoustic waves. When data acquired by the probes 602 is reconstructed by the information processing unit, the acquisition of a three-dimensional photoacoustic image is allowed. Note that ultrasound wave echo measurement used to acquire photoacoustic characteristics inside the object is performed by a linear ultrasound wave probe 604. The linear ultrasound wave probe 604 is capable of performing scanning with the hemispherical container 601.

The measurement light emitted from the light irradiation unit 603 is irradiated onto the object via the space 605. In order to effectively generate photoacoustic waves, it is necessary to irradiate the light in a substantially short period of time according to the heat characteristics of the object. When the object is a living body, the pulsed light emitted from the light source preferably has a pulse width of 10 to 50 nanoseconds. Here, the light irradiation unit 603 uses a titanium sapphire laser serving as a solid-state laser. In addition, in order to measure an oxygen saturation degree, light having two wavelengths of 760 nm and 800 nm is used.

The probes 602 receive the photoacoustic waves and convert the same into an electrical signal. After that, the probes 602 output the electrical signal to a signal processing unit (not shown). Here, CMUTs (Capacitive Micro-Machined Ultrasonic Transducers) are used as the probes 602. The probes are single devices, have an opening with a diameter φ of 3 mm, and have a band of 0.5 MHz to 5 MHz. Since the probes have a low frequency band, it is possible to acquire a fine image even from vessels having a thickness of about 3 mm. That is, a situation in which vessels are voided to look like a ring shape hardly occurs.

The signal processing unit performs the signal processing of the electrical signal output from the probes 602 and performs 2048 sampling at a sampling frequency of 40 MHz. In addition, data is 12-bit data with a code.

The linear ultrasound wave probe 604 is a transceiver that transmits ultrasound waves to the object and outputs an electrical signal after receiving echo waves reflected by the object. As such a device, a PZT (Piezoelectric Ceramics) is used. The linear ultrasound wave probe 604 has 256 devices and a band of 5 MHz to 10 MHz. In addition, the linear ultrasound wave probe 604 performs 2048 sampling at a sampling frequency of 40 MHz. In addition, data is 12-bit data with a code.

Note that the photoacoustic waves attenuate in the course of propagation before reaching the probes after the generation. Therefore, the attenuation is preferably corrected. That is, initial sound pressure generated by absorbers as expressed by formula (1) attenuates before reaching the probes. Formula (5) shows the relationship between initial sound pressure (Pᵢ) and sound pressure (P_{d}) detected by a detector.

[Math. 6]

P_{d}=PᵢEXP(−αfL) (6)

Here, α is an attenuation coefficient, Pᵢ is initial sound pressure, f is a transmission frequency, and L is a propagation distance. As described above, the photoacoustic waves attenuate exponentially. Therefore, in order to improve accuracy in calculating an optical coefficient, it is necessary to correct the attenuation.

In addition, the processing apparatus of the third embodiment includes an information processing unit, a light control unit, a signal processing unit, and an apparatus control unit not shown in FIGS. 6A and 6B. Since the functions of the respective units are the same as those of the first embodiment, their descriptions will be omitted.

(Processing Flow)

A description will be particularly given, with reference to the flowchart of FIG. 5, of a part different from that of the second embodiment.

When measurement is started in step S501, a breast is placed on the holding member 606.

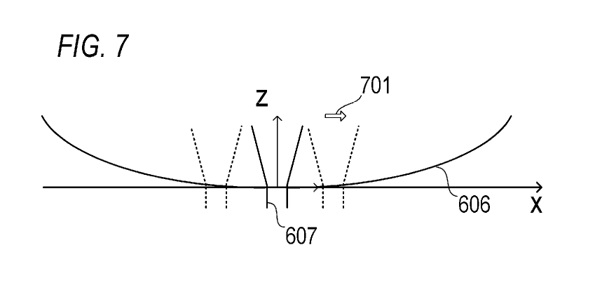

In step S502, photoacoustic measurement is performed. FIG. 7 is a schematic view showing a state in which the photoacoustic measurement is performed while scanning a position at which the irradiation light 607 is incident on the object. The position on which the light is incident sequentially moves to a direction indicated by an arrow 701. The light travels in the acoustic matching material while maintaining its almost parallel state. However, after being incident on the inside of the object accommodated in the holding member 606, the light scatters inside the object according to a scattering coefficient. The probes 602 receive photoacoustic waves generated inside the object.

Note that in step S502, ultrasound wave measurement is further performed after the photoacoustic measurement. In this case, the linear ultrasound wave probe 604 is scanned in the X direction. The information processing unit generates ultrasound wave image data indicating acoustic impedance inside the object based on an electrical signal output from the ultrasound wave probe 604. As a result, it is possible to acquire a B-scan image parallel to a ZY plane. In addition, an attenuation specific value is calculated from the B-scan image, and the attenuation of the photoacoustic waves inside the object is corrected. At this time, the information processing unit operates as a sixth acquirer of the present invention.

In step S503, the information processing unit extracts vessels from a photoacoustic image for each pulse. At this time, the information processing unit selects vessels having a thickness of 0.5 mm to 3 mm from among the vessels using a filter or the like.

In step S504, the information processing unit generates a two-dimensional signal intensity distribution and calculates specific information distances as respective characteristic values in an irradiation direction and a perpendicular direction. Here, as shown in FIG. 7, signal intensity is projected in a Y axis at each height Z to acquire a two-dimensional intensity image of a ZX plane. In the two-dimensional intensity image, the respective specific information distances are calculated from an envelope line in the irradiation direction (Z direction) and the perpendicular direction (X direction). On this occasion, a maximum value may be projected with the origin of the two-dimensional intensity image for each irradiation position of the light set at the same position to generate the two-dimensional intensity image. The two-dimensional signal intensity distribution contains information associated with each of an absorption coefficient contributing to an invasive depth in the irradiation direction and a scattering coefficient contributing also to the spread of the light in the vertical direction. For example, the light travels straight when the light does not scatter at all. Therefore, the light does not spread in the perpendicular direction (in-plane direction perpendicular to a light axis, i.e., the X direction in FIG. 7). That is, since the light does not reach, a photoacoustic signal does not output from such a region. Conversely, when the light scatters, it is possible to acquire a signal from a region spreading from the light axis in the perpendicular direction. In addition, since both the absorption coefficient and the scattering coefficient contribute to the invasive length, the invasive length may not be simply divided.

Here, the spread in the perpendicular direction is calculated depending on to what degree a signal having a value greater than a threshold reaches in the X direction at a certain depth. Since the information reflects a scattering coefficient, it is possible to calculate the scattering coefficient. In addition, it is possible to calculate an effective attenuation coefficient from the specific information distance in the irradiation direction. An effective attenuation coefficient μ_{eff} has an absorption coefficient μₐ and a scattering coefficient μₛ as parameters as in the above formula (5). As a result, it is possible to calculate the absorption coefficient μₐ with the scattering coefficient μₛ and the effective attenuation coefficient μ_{eff} calculated from an image.

When the light source is a line light source or a surface light source, integration processing is performed after processing associated with scattering and absorption as described above is performed for each position at which the light is incident on the object.

In step S505, the information processing unit (not shown) retrieves the database. The database accumulates the correspondence between the specific information distance in the irradiation direction and the effective attenuation coefficient μ_{eff} and the correspondence between the specific information distance in the perpendicular direction and the scattering coefficient μₛ.

When the closest specific information distance is found in step S506, the corresponding scattering coefficient μₛ and the effective attenuation coefficient μ_{eff} are determined as background optical coefficients. In addition, the information processing unit calculates the absorption coefficient μₐ from the scattering coefficient μₛ and the effective attenuation coefficient μ_{eff}. Note that in the third embodiment, the specific information distances are used as indexes to indicate the spread of the signal intensity in the irradiation direction and the perpendicular direction. However, other calculation methods may be used. For example, it may also be possible to read the spread in each direction from a two-dimensional signal intensity distribution image. As described above, the spread in the perpendicular direction of the two-dimensional signal intensity distribution image has a certain correlation with the scattering coefficient, and the spread in the irradiation direction thereof has a certain correlation with the absorption coefficient. Because of this, it may also be possible to acquire the absorption coefficient based on a correspondence with the first specific information distribution in the irradiation direction among the first specific information distributions and acquire the scattering coefficient based on a correspondence with the first specific information distribution in the perpendicular direction among the specific information distributions. In addition, for example, a multiplicity of correspondences between two-dimensional signal intensity distribution images and background optical coefficients may be prepared in advance in the database and verified by pattern recognition against images in the database to acquire background optical coefficients.

The measurement is finished in step S507.

As described above, according to the present invention, it is possible to calculate background optical coefficients such as the absorption coefficient and the scattering coefficient of light inside an object using data calculated from a photoacoustic image.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-51615, filed on Mar. 15, 2016, which is hereby incorporated by reference herein in its entirety.

What is claimed is:

1. A processing apparatus comprising:
a first acquirer configured to acquire a first specific information distribution of an object based on an electrical signal acquired by receiving acoustic waves propagating from the object onto which light is irradiated;
a second acquirer configured to acquire a characteristic value of the first specific information distribution of the object;
a third acquirer configured to acquire information indicating a correspondence between an optical coefficient and the characteristic value of the first specific information distribution; and
a fourth acquirer configured to acquire the optical coefficient of the object using the characteristic value of the first specific information distribution of the object and the information indicating the correspondence.

2. The processing apparatus according to claim 1, wherein
the characteristic value includes a value of the first specific information in an irradiation direction in which the light is irradiated onto the object and a value of the first specific information in a different direction different from the irradiation direction, the values being associated with an absorption coefficient and a scattering coefficient contained in the optical coefficient of the object.

3. The processing apparatus according to claim 2, wherein
the fourth acquirer is configured to:
acquire the absorption coefficient using the characteristic value of the first specific information distribution in the irradiation direction and the information indicating the correspondence, and
acquire the scattering coefficient using the characteristic value of the first specific information distribution in the different direction and the information indicating the correspondence.

4. The processing apparatus according to claim 2, wherein
the different direction is a direction substantially orthogonal to the irradiation direction.

5. The processing apparatus according to claim 1, further comprising:
a fifth acquirer configured to acquire a light intensity distribution of the light inside the object using the optical coefficient acquired by the fourth acquirer and acquire a second specific information distribution inside the object using the light intensity distribution.

6. The processing apparatus according to claim 5, wherein
the fifth acquirer is configured to acquire the second specific information distribution using the electrical signal used to acquire the first specific information distribution and the light intensity distribution.

7. The processing apparatus according to claim 5, wherein
the fifth acquirer is configured to correct the first specific information distribution using the light intensity distribution to acquire the second specific information distribution.

8. The processing apparatus according to claim 5, wherein
the fifth acquirer is configured to acquire the second specific information distribution using an electrical signal different from the electrical signal used to acquire the first specific information distribution and the light intensity distribution.

9. The processing apparatus according to claim 5, wherein
the first specific information includes one of initial sound pressure and optical energy absorption density, and
the second specific information includes an absorption coefficient.

10. The processing apparatus according to claim 1, further comprising:
a transceiver configured to transmit ultrasound waves to the object and receive echo waves reflected by the object to output a second electrical signal; and
a sixth acquirer configured to generate ultrasound wave image data relating to the inside of the object based on the second electrical signal and corrects attenuation of the acoustic waves inside the object based on the ultrasound wave image data.

11. The processing apparatus according to claim 1, wherein
the correspondence includes statistical data acquired in advance from a plurality of objects.

12. The processing apparatus according to claim 1, further comprising:
a memory configured to store the information indicating the correspondence, wherein
the third acquirer is configured to read the information indicating the correspondence from the memory to acquire the information indicating the correspondence.

13. The processing apparatus according to claim 1, wherein
the first acquirer is configured to perform image reconstruction using the electrical signal to acquire the first specific information distribution of the object.

14. A photoacoustic apparatus comprising:
the processing apparatus according to claim 1;
a light source configured to irradiate the light onto the object; and
a probe configured to receive the acoustic waves propagating from the object onto which the light is irradiated to output the electrical signal.

15. A processing method comprising:
a first acquisition step of acquiring a first specific information distribution of an object based on an electrical signal acquired by receiving acoustic waves propagating from the object onto which light is irradiated;
a second acquisition step of acquiring a characteristic value of the first specific information distribution of the object;
a third acquisition step of acquiring information indicating a correspondence between an optical coefficient and the characteristic value of the first specific information distribution; and
a fourth acquisition step of acquiring the optical coefficient of the object using the characteristic value of the first specific information distribution of the object and the information indicating the correspondence.

16. A non-transitory computer readable storing medium recording a computer program for causing a computer to perform a method comprising the steps of:
acquiring a first specific information distribution of an object based on an electrical signal acquired by receiving acoustic waves propagating from the object onto which light is irradiated;
acquiring a characteristic value of the first specific information distribution of the object;
acquiring information indicating a correspondence between an optical coefficient and the characteristic value of the first specific information distribution; and
acquiring the optical coefficient of the object using the characteristic value of the first specific information distribution of the object and the information indicating the correspondence.
